(19) United States
(12) Patent Application Publication
(10) Pub. No.: US 2005/0266055 A1
(43) Pub. Date: Dec. 1, 2005

(54) Low-viscosity cosmetic or dermatological preparations

(75) Inventors: Sabine Stiller, Hamburg (DE); Jens Schulz, Hamburg (DE); Rolf Daniels, Salzgitter (DE); Ingo Hahn, Pinneberg (DE); Stephanie Von Der Fecht, Schenenefeld (DE); Birgit Groteluschen, Wildeshausen (DE)

Correspondence Address: ALSTON & BIRD LLP;BANK OF AMERICA PLAZA, 101 SOUTH TRYON STREET, SUITE 4000, CHARLOTTE, NC, 28280-4000, US

(73) Assignee: Beiersdorf AG, Hamburg (DE)

(21) Appl. No.: 11/064,214

(22) Filed: Feb. 23, 2005

Related U.S. Application Data
(63) Continuation of application No. PCT/EP03/09024, filed on Aug. 14, 2003.

(30) Foreign Application Priority Data
Aug. 23, 2002 (DE) 102 38 649.8

(51) Int. Cl. A61K 9/70; A61K 7/42; A61K 7/06; A61K 7/11

(52) U.S. Cl. 424/443; 424/70.13; 424/59

(57) ABSTRACT

A low-viscosity Pickering water-in-oil emulsion having a viscosity of less than 3000 mPa·s, comprising (1) an oil phase, (2) a water phase, (3) at least one type of microfine, inorganic metal oxide particles a) having an average particle size of less than 200 nm, b) exhibiting both hydrophilic and also lipophilic properties, c) dispersible both in lipophilic and also in hydrophilic liquids and d) optionally surface-coated, (4) 0.01 to 5% by weight of at least one polymeric nonionic and/or cationic thickener and (5) at most 0.5% by weight of one or more emulsifiers.

### CROSS-REFERENCE TO RELATED APPLICATIONS

This is a continuation application of PCT/EP03/09024, filed Aug. 14, 2003, which is incorporated herein by reference in its entirety, and also claims the benefit of German Application No. 102 38 649.8, filed Aug. 23, 2002.

### FIELD OF THE INVENTION

The present invention relates to low-viscosity, emulsifier-free finely disperse cosmetic and dermatological preparations of the oil-in-water type, in particular emulsifier-free O/W emulsions which have a viscosity of less than 3000 mPa·s, and to their use for cosmetic and medicinal purposes. In addition, the present invention relates to cosmetic and dermatological wipes which are impregnated with such preparations.

### BACKGROUND OF THE INVENTION

Emulsions are generally understood as meaning heterogeneous systems which consist of two liquids which are immiscible or have only limited miscibility with one another, which are usually referred to as phases. In an emulsion, one of the two liquids is dispersed in the form of very fine droplets in the other liquid.

If the two liquids are water and oil and if oil droplets are finely dispersed in water, then this is an oil-in-water emulsion (O/W emulsion, e.g., milk). The basic character of a O/W emulsion is defined by the water. In a water-in-oil emulsion (W/O emulsion, e.g., butter), the principle is reversed, the basic character here being determined by the oil.

In order to achieve permanent dispersion of one liquid in another, emulsions in the traditional sense require the addition of an interface-active substance (emulsifier). Emulsifiers have an amphiphilic molecular structure, consisting of a polar (hydrophilic) and a nonpolar (lipophilic) molecular moiety, which are spatially separate from one and a nonpolar (lipophilic) molecular moiety, which are spatially separate from one another. In simple emulsions, finely disperse droplets of one phase, surrounded by an emulsifier shell, (water droplets in W/O emulsions or lipid vesicles in O/W emulsions) are present in the second phase. Emulsifiers lower the interfacial tension between the phases by positioning themselves at the interface between two liquids. At the oil/water phase boundary they form interfacial films, which prevent irreversible coalescence of the droplets. Emulsions are frequently stabilized using emulsifier mixtures.

Traditional emulsifiers can, depending on their hydrophilic molecular moiety, be divided into ionic (anionic, cationic and amphoteric) and nonionic:
The most well known example of an anionic emulsifier is soap, which is usually the term used for the water-soluble sodium or potassium salts of saturated and unsaturated higher fatty acids.

Important representatives of cationic emulsifiers are the quaternary ammonium compounds.

The hydrophilic molecular moiety of nonionic emulsifiers often consists of glycerol, polyglycerol, sorbitans, carbohydrates and polyoxyethylene glycols and, in most cases, is linked to the lipophilic molecular moiety via ester and ether bonds. The lipophilic molecular moiety usually consists of fatty alcohols, fatty acids or iso fatty acids.

By varying the structure and the size of the polar and nonpolar molecular moiety, the lipophilicity and hydrophilicity of emulsifiers can be varied within wide limits.

A decisive factor for the stability of an emulsion is the correct choice of emulsifiers. The characteristics of all substances present in the system are to be taken into consideration here. In the case of, for example, skincare emulsions, polar oil components such as, for example, UV filters can lead to instabilities. Besides the emulsifiers, therefore, other stabilizers are also used which, for example, increase the viscosity of the emulsion and/or act as protective colloid.

Emulsions represent an important type of product in the field of cosmetic and/or dermatological preparations which is used in very diverse fields of application. For example, there is a large number of products—such as lotions and creams—available for the care of the skin, in particular for the regreasing of dry skin. One aim of skincare is to compensate for the loss by the skin of grease and water caused by daily washing. Furthermore, skincare products should protect against environmental influences—in particular against sun and wind—and delay skin aging.

Cosmetic emulsions are also used as deodorants. Such formulations serve to eliminate body odor which is produced when fresh perspiration, which is in itself odorless, is decomposed by microorganisms.

For the cleansing of the skin and skin appendages too, emulsions are used in the form of cleansing emulsions. They are mostly used for cleansing the face and in particular for removing decorative cosmetics. In contrast to other cleansing preparations, such as, for example, soap, such cleansing emulsions have the advantage of being particularly skin-compatible since they can comprise care oils and/or nonpolar active ingredients—such as, for example, vitamin E—in their lipophilic phase.

A particular application form of cosmetic and/or dermatological emulsions is their use as impregnation solution for cosmetic or dermatological wipes.

A disadvantage of the prior art is the fact that emulsions generally comprise organic emulsifiers which stabilize the emulsions or permit their preparation in the first place. In itself, the use of emulsifiers in cosmetic or dermatological preparations is acceptable. Nevertheless, emulsifiers, like ultimately every chemical substance, can in specific cases cause allergic reactions or reactions based on user hypersensitivity. Undesired secondary effects which may arise in sensitive people are, for example, skin irritation. There has therefore been no lack of attempts to reduce the amount of customary emulsifiers to a minimum, and in the ideal case even completely.

A reduction in the required amount of emulsifier can be achieved, for example, if the fact that certain very finely divided solid particles have an emulsion-stabilizing effect is exploited. Here, the solid substance accumulates at the oil/water phase boundary in the form of a layer, as a result of which coalescence of the disperse phases is prevented. Of particular importance here are, in particular, the surface properties of the solid particles.

These so-called Pickering emulsions generally have a high viscosity of more than 8000 mPa·s. Flowable (with a viscosity of about 3000 mPa·s to about 8000 mPa·s) or even low-viscosity (with a viscosity of less than 3000 mPa·s) O/W Pickering emulsions could not be prepared by the prior art.

Emulsions with a very low viscosity (low-viscosity or sprayable emulsions) have hitherto only been able to be formulated in a very involved process, if at all. Accordingly, the supply of formulations of this type is extremely low. Nevertheless, such formulations could offer the consumer as yet unknown cosmetic effects.

European patent specification 667 144 does describe cosmetic sunscreen compositions which represent oil-in-water emulsions and comprise inorganic nanopigments based on metal oxides as photoprotective agents, where the formulations can also be sprayable. These preparations represent so-called PIT emulsions which are prepared by phase reversal and are therefore in particularly finely disperse form. However, this specification was unable to point the way to the present invention.

Anyway, low viscosity preparations of the prior art often have the disadvantage that they are restricted to a narrow field of application or a limited choice of raw materials. The incorporation of relatively high concentrations of polar oil components also often presents difficulties. In some cases, however, it is desired to incorporate large amounts of polar oil components into a formulation, for example in order to dissolve solid UV filter substances and thus to be able to achieve a high light protection factor.

### SUMMARY OF THE INVENTION

One object of the present invention was to prepare emulsifier-free preparations of the oil-in-water type which have a very low viscosity and do not have the disadvantages of the prior art. It was a further object of the invention to discover routes to cosmetic or dermatological O/W emulsions with as low a viscosity as possible which are stable to increased electrolyte concentrations and into which it is possible to incorporate large amounts of polar oil components. In addition, it was an object of the invention to find a method of stabilizing low-viscosity O/W formulations.

It was also an object of the present invention to provide products with the broadest application diversity possible. For example, it was the aim to provide bases for preparation forms such as cleansing emulsions, face and bodycare preparations or deodorants, but also distinctly medicinal-pharmaceutical administration forms, for example preparations to combat acne and other skin symptoms. In addition, the aim was also to find preparations which are suitable as impregnation solutions for cosmetic or dermatological wipes.

Impregnated wipes are articles of daily need and are used in very diverse areas. Inter alia, they allow efficient and skin-friendly cleansing and care, particularly also in the absence of (running) water.

The actual article of use consists here of two components:
a) a dry wipe which is made of material such as paper and/or very diverse mixtures of natural or synthetic fibers and
b) a low-viscosity impregnation solution.

Cosmetic or dermatological wipes can consist either of water-soluble materials (e.g. such as toilet paper) or of water-insoluble materials. In addition, the wipes may be smooth, perforated or else surface-structured.

Conventional impregnation solutions for water-insoluble nonwoven materials have hitherto often had the shortcoming of low long-term stability. Such emulsions have a tendency, particularly at elevated ambient temperature, toward phase separation, which is a decisive disadvantage for the impregnation process and for the final quality of the end product.

The long-term stability of impregnation solutions of the prior art is generally ensured through the use of increased emulsifier concentrations and considerable energy input—for example during homogenization.

It was therefore an object of the present invention to find impregnation solutions with long-term stability for applying to water-insoluble nonwoven materials which do not have the disadvantages of the prior art and which, even if the contents of the emulsifier are low or they are in the form of emulsifier-free formulations, represent low-viscosity O/W emulsions with long-term stability which do not have to be homogenized as far as possible.

It was surprising and in no way foreseeable by the person skilled in the art that low-viscosity Pickering emulsions which represent finely disperse systems of the water-in-oil type and have a viscosity of less than 3000 mPa·s comprising
(1) an oil phase,
(2) a water phase,
(3) at least one type of microfine, inorganic particle chosen from the group of metal oxides which
   a) have an average particle size of less than 200 nm,
   b) exhibit both hydrophilic and also lipophilic properties,
   c) are dispersible both in lipophilic and also in hydrophilic liquids and
   d) are optionally surface-coated,
(4) 0.01 to 5% by weight of at least one polymeric nonionic and/or cationic thickener and
(5) at most 0.5% by weight of one or more emulsifiers,
   overcome the disadvantages of the prior art.

According to the invention, it is particularly advantageous if the preparations comprise significantly less than 0.5% by weight of one or more emulsifiers. Very particular preference is given to preparations according to the invention which are entirely free from emulsifiers in the conventional sense.

According to the invention, it is also particularly advantageous if the preparations have a viscosity of less than 2000 mPa·s.

### DETAILED DESCRIPTION OF THE PREFERRED EMBODIMENTS

The preparations according to the invention are extremely satisfactory preparations in every respect which have a significantly lower viscosity than conventional Pickering emulsions and are therefore especially suited to use as bases for preparation forms with diverse application purposes. It was particularly surprising that, by combining microfine inorganic particles according to the invention with nonionic and/or cationic thickeners, low-viscosity preparations are accessible, whereas the use of anionic thickeners destroys the emulsion structure.

The preparations according to the invention represent an enrichment of the prior art with regard to low-viscosity O/W emulsions in every respect.

Pickering emulsions of the prior art (also called primary emulsions below) are obtainable by dispersing amphiphilic particles suitable for preparing Pickering emulsions in one of the two emulsion phases. The phases are advantageously combined with homogenization using Beco mixers or other suitable homogenization plants. The homogenization can take place either cold (i.e. at less than 30° C.) or hot (i.e. at more than 60° C.).

It was surprising and unforeseeable that flowable and low-viscosity preparations according to the invention are obtainable by subsequent dilution of thickener-free Pickering emulsions of the prior art, i.e. by dilution of high-viscosity primary emulsions which comprise no thickeners. In addition, these are mixed with an up to twenty-fold amount of a dilution solution which comprises, in an aqueous base, 0.01 to 5% by weight of one or more nonionic and/or cationic thickeners according to the invention.

The invention thus also provides a method of preparing O/W Pickering emulsions with a viscosity of less than 3000 mPa·s, characterized in that an O/W primary emulsion which comprises
(1) an oil phase,
(2) a water phase,
(3) at least one type of microfine inorganic particle chosen from the group of metal oxides which
   a) have an average particle size of less than 200 nm,
   b) exhibit both hydrophilic and also lipophilic properties,
   c) are dispersible both in hydrophilic and also in lipophilic media,
      and which has a viscosity of 8000 mPa·s or more is mixed with an up to twenty-fold amount of an aqueous solution in which a suitable thickener is present in an evenly distributed form.

It is also possible for the purposes of the present invention to add the aqueous dilution medium (without added thickener) and the thickener separately to the Pickering primary emulsion provided uniform thorough mixing—for example by one of the methods specified below—can be achieved.

The mixing can advantageously take place by stirring (e.g. by means of paddle stirrers) at an average stirring speed of more than 50 rpm or else also by vigorous shaking in a suitable vessel which, to improve thorough mixing, can advantageously also contain mixing elements such as, for example, plastic balls.

For the purposes of the present invention, the primary emulsion can be diluted irrespective of the point in time when it has been prepared.

Polymeric thickeners for the purposes of the present invention are macromolecules which have a largely linear structure and have intermolecular forces of interaction, which permit secondary and primary valence bonds between the individual molecules and thus the formation of a reticular structure. Some are water-soluble, water-dispersible natural or synthetic polymers which, in aqueous systems, form gels or viscous solutions. They increase the viscosity of the water by either binding water molecules (hydration) or else by absorbing and encapsulating water into their interwoven macromolecules, at the same time restricting the mobility of the water.

The nonionic and cationic polymeric thickeners listed below are, for example, advantageous according to the invention:
organic modified natural substances, such as, for example, hydroxyethylcellulose and other cellulose ethers, hydroxyethylcellulose and hydroxypropylcellulose and the like, and modified starch derivatives
organic natural compounds, such as, for example, agar agar, carrageen, polyoses, starch, dextrins, gelatin, caseine,
organic completely synthetic compounds, such as, for example, vinyl polymers, polyethers, polyimines, polyamides and derivatives of polyacrylic acid,
inorganic compounds, such as, for example, polysilicic acids, clay minerals such as montmorillonite, zeolites, silicas.

Of advantage for the purposes of the present invention are, for example, alkylcelluloses, which is the name for the alkyl ethers of cellulose. They are characterized by the following structural formula 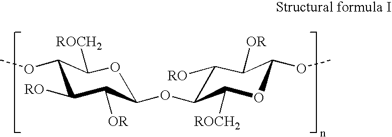
in which R may be a hydrogen or an alkyl group having 1 to 4 carbon atoms.

An alkylcellulose preferred according to the invention is hydroxyethylcellulose (CAS 9004-62-0), for example that available under the trade name Natrosol hydroxyethylcellulose from Aqualon.

Of particular advantage for the purposes of the present invention are also cellulose mixed ethers, which contain, in addition to a dominant content of methyl groups, also 2-hydroxyethyl, 2-hydroxypropyl or 2-hydroxybutyl groups. Particular preference is given to (hydroxypropyl)methylcelluloses, for example those available under the trade name Methocel E4M from Dow Chemical Comp.

Advantageous cationic thickeners for the purposes of the present invention are also cationically modified polyacrylic acid derivatives, in particular those which consist of homopolymers of methacryloylethyltrimethylammonium chloride (INCI: Polyquaternium 37), for example available under the name Synthalen CR from FA 3V Sigma.

The total amount of one or more polymeric thickeners in the finished cosmetic or dermatological O/W Pickering emulsions is advantageously chosen to be less than 5.0% by weight, preferably between 0.05 and 3% by weight, in each case based on the total weight of the preparations.

It is also advantageous, although not obligatory, for the Pickering emulsions according to the invention to comprise further auxiliaries which can additionally increase the stability of these preparations, for example substances which are chosen from the group of waxes and/or oil thickeners, and of electrolytes.

It is also advantageous for the Pickering emulsions according to the invention to comprise auxiliaries which can contribute to reducing or preventing a dull or dry feel on the skin following their application, the main purpose of these substances possibly being a different one. Preferably, the substances are, for example, chosen from the group of asymmetrically substituted S-triazine derivatives, cyclodextrins, film formers and polymeric moisturizers, it being possible for these substances to be present either individually or in a mixture.

The cosmetic properties of the Pickering emulsions according to the invention can additionally, for example, be further improved by also using oils in the oil phase which have a viscosity of less than 30 mPa·s, in particular less than 20 mPa·s (determined using a rheometer from Contraves (Rheomat 108E) at a shear gradient of 500/s and a temperature of 25° C.).

The cosmetic properties (in particular the wetting properties and the skin feel) of the Pickering emulsions according to the invention can additionally also be further improved through the use of chitosan (in concentrations of less than 1% by weight, in particular 0.1 to 0.5% by weight) and/or other cationic film formers, such as, for example, starch hydroxypropyltrimonium chloride, which is available under the trade name Sensomer CI-50 from Ondeo Nalco.

It is also advantageous if the Pickering emulsions according to the invention comprise solubility promoters; PPG-15 stearyl ether and PEG-40 hydrogenated castor oil are particularly advantageous.

Microfine Inorganic Particles:

The amphiphilic character of the microfine particles according to the invention is evident, for example, from the fact that they are dispersible both in hydrophilic liquids and also in lipophilic liquids. Accordingly, pigments which are characterized by the manufacturer as being hydrophobic can also have stabilizing properties.

Wettability determinations are suitable for allowing more precise qualification of the surface properties. Here, the sedimentation behavior of the pigments in ethanol/water mixtures of varying concentration is observed in order to make it possible to estimate the stabilization properties of pigments. For this purpose, the time which a pigment requires to immerse completely into a liquid is measured. Wettable pigments are those which are completely immersed into a liquid within 10 min. For stabilizing Pickering emulsions pigments which are wettable by a 40% strength ethanol/water mixture are suitable.

It is advantageous to choose the average particle diameter of the particles used to be between 1 nm and 200 nm, particularly advantageously between 5 nm and 100 nm.

It is also advantageous to choose the concentration of all amphiphilic particles according to the invention to be greater than 0.1% by weight, advantageously between 0.1% by weight and 30% by weight, particularly advantageously between 1% by weight and 15% by weight, in each case based on the total weight of the preparation.

For the purposes of the present invention, advantageous particles are all those which are suitable for stabilizing low-viscosity Pickering O/W emulsions. It is essentially insignificant for the present invention in which of the potentially naturally occurring modifications the particles are present.

For stabilizing the Pickering emulsions preference is given to using untreated, virtually pure pigment particles, in particular those which can also be used as dye in the food industry and/or as absorber of UV radiation in sunscreen compositions. Examples of advantageous pigments are the titanium dioxide pigments available from Merck and Tayca, and the zinc oxide pigments available from Tayca and BASF, and those which are available under the trade names Zinkoxid neutral from Haarmann & Reimer or NanoX from Harcros Chemical Group.

For the purposes of the present invention, Pickering emulsions are likewise advantageously stabilized by inorganic pigments which have been surface-treated (“coated”) to repel water, the intention being at the same time to form or retain the amphiphilic character. This surface treatment can consist in providing the pigments with a thin hydrophobic layer by methods known per se.

According to the invention, the pigments can be advantageously surface-treated (“coated”), where, for example, the intention is to form or retain a hydrophilic, amphiphilic or hydrophobic character. This surface treatment can consist in providing the pigments with a thin hydrophilic and/or hydrophobic inorganic and/or organic layer by methods known per se. The various surface coatings can also comprise water for the purposes of the present invention.

Inorganic surface coatings for the purposes of the present invention can consist of aluminum oxide (Al₂O₃), aluminum hydroxide Al(OH)₃, or aluminum oxide hydrate (also: alumina, CAS No.: 1333-84-2), sodium hexametaphosphate (NaPO₃)₆, sodium metaphosphate (NaPO₃)ₙ, silicon dioxide (SiO₂) (also: silica, CAS No.: 7631-86-9), or iron oxide (Fe₂O₃). These inorganic surface coatings may be present on their own, in combination and/or in combination with organic coating materials.

Organic surface coatings for the purposes of the present invention can consist of vegetable or animal aluminum stearate, vegetable or animal stearic acid, lauric acid, dimethylpolysiloxane (also: dimethicone), methylpolysiloxane (methicone), simethicone (a mixture of dimethylpolysiloxane with an average chain length of from 200 to 350 dimethylsiloxane units and silica gel) or alginic acid. These organic surface coatings can be present on their own, in combination and/or in combination with inorganic coating materials.

Zinc oxide particles and predispersions of zinc oxide particles suitable according to the invention are available under the following trade names from the companies listed:

| Trade name | Coating | Manufacturer |
|---|---|---|
| Z-Cote HP1 | Dimethicone | BASF |
| Z-Cote | / | BASF |
| ZnO NDM | Dimethicone | H&R |
| NanoX | / | Harcros Chemical |
| Zinkoxid | / | H&R |
| neutral | | |
| MZ-707S | Methicone | Tayca Corporation |
| MZ-707M | Dimethicone | Tayca Corporation |

Suitable titanium dioxide particles and predispersions of titanium dioxide particles are available under the following trade names from the companies listed:

| Trade name | Coating | Manufacturer |
|---|---|---|
| Eusolex T- | Alumina/Simethicone | Merck KgaA |
| 2000 | | |
| MT-100TV | Alumina/stearic acid | Tayca Corporation |
| MT-100Z | Alumina/stearic acid | Tayca Corporation |
| UV-Titan X610 | Alumina/dimethicone | Kemira |
| MT-150A | / | Tayca Corporation |
| MT-500B | / | Tayca Corporation |
| MT-100SA | Alumina/silica | Tayca Corporation |
| MT-01 | Alumina/stearic acid | Tayca Corporation |
| Titandioxid | Octyltrimethylsilane | Degussa |
| T805 (Uvinul | | |
| TiO₂) | | |
| Eusolex T- | Water/alumina/sodium | Merck |
| aqua | metaphosphate | |

For the purposes of the present invention, titanium dioxides which are coated with simethicone and alumina, where the coating can also comprise water, are very particularly advantageous. One example of this is the titanium dioxide available under the trade name Eusolex T2000 from Merck.

Also advantageous for the purposes of the present invention is the use of a mixture of different types of pigment both within a crystal, for example as iron mixed oxide, and also by combining two or more types of pigment within a preparation.

It is also advantageous, although not obligatory, to combine the microfine particles according to the invention with further amphiphilic particles which may optionally also contribute to the stabilization of the Pickering emulsions.

The oil phase of the formulations according to the invention is advantageously chosen from the group of polar oils, for example from the group of lecithins and of fatty acid triglycerides, namely the triglycerol esters of saturated and/or unsaturated, branched and/or unbranched alkanecarboxylic acids with a chain length of from 8 to 24, in particular 12 to 18, carbon atoms. The fatty acid triglycerides can, for example, be chosen advantageously from the group of synthetic or semisynthetic and natural oils, such as, for example, cocoglyceride, olive oil, sunflower oil, soya oil, peanut oil, rapeseed oil, almond oil, palm oil, coconut oil, castor oil, wheatgerm oil, grapeseed oil, thistle oil, evening primrose oil, macadamia nut oil and the like.

Also advantageous according to the invention are, for example, natural waxes of animal and vegetable origin, such as, for example, beeswax and other insect waxes, and berry wax, shea butter and/or lanolin (wool wax).

For the purposes of the present invention, further advantageous polar oil components can also be chosen from the group of esters of saturated and/or unsaturated, branched and/or unbranched alkanecarboxylic acids with a chain length of from 3 to 30 carbon atoms and saturated and/or unsaturated, branched and/or unbranched alcohols with a chain length of from 3 to 30 carbon atoms, and from the group of esters of aromatic carboxylic acids and saturated and/or unsaturated, branched and/or unbranched alcohols with a chain length of from 3 to 30 carbon atoms. Such ester oils can then advantageously be chosen from the group consisting of octyl palmitate, octyl cocoate, octyl isostearate, octyl dodecyl myristate, octyldodecanol, cetearyl isononanoate, isopropyl myristate, isopropyl palmitate, isopropyl stearate, isopropyl oleate, n-butyl stearate, n-hexyl laurate, n-decyl oleate, isooctyl stearate, isononyl stearate, isononyl isononanoate, 2-ethylhexyl palmitate, 2-ethylhexyl laurate, 2-hexyldecyl stearate, 2-octyidodecyl palmitate, stearyl heptanoate, oleyl oleate, oleyl erucate, erucyl oleate, erucyl erucate, tridecyl stearate, tridecyl trimellitate, and synthetic, semisynthetic and natural mixtures of such esters, such as, for example, jojoba oil.

The oil phase can also advantageously be chosen from the group of dialkyl ethers and dialkyl carbonates, advantageous examples being dicaprylyl ether (Cetiol OE) and/or dicaprylyl carbonate, for example that available under the trade name Cetiol CC from Cognis.

It is also preferred to select the oil component or the oil components from the group consisting of isoeicosane, neopentyl glycol diheptanoate, propylene glycol dicaprylate/dicaprate, caprylic/capric/diglyceryl succinate, butylene glycol dicaprylate/dicaprate, C₁₂₋₁₃-alkyl lactate, di-C₁₂₋₁₃-alkyl tartrate, triisostearin, dipentaerythrityl hexacaprylate/hexacaprate, propylene glycol monoisostearate, tricaprylin, dimethylisosorbide. It is particularly advantageous if the oil phase of the formulations according to the invention has a content of C₁₂₋₁₅-alkyl benzoate, or consists entirely of this.

Advantageous oil components are also, for example, butyloctyl salicylate (for example that available under the trade name Hallbrite BHB from CP Hall), hexadecyl benzoate and butyloctyl benzoate and mixtures thereof (Hallstar AB) and/or diethylhexyl naphthalate (Hallbrite TQ or Corapan TQ from H&R).

Any desired mixtures of such oil and wax components can also be used advantageously for the purposes of the present invention.

In addition, the oil phase can likewise advantageously also comprise non-polar oils, for example those which are chosen from the group of branched and unbranched hydrocarbons and hydrocarbon waxes, in particular mineral oil, vaseline (petrolatum), paraffin oil, squalane and squalene, polyolefins, hydrogenated polyisobutenes and isohexadecane. Among the polyolefins, polydecenes are the preferred substances.

The oil phase can also advantageously have a content of cyclic or linear silicone oils or consist entirely of such oils, although it is preferred to use an additional content of other oil phase components apart from the silicone oil or the silicone oils.

Silicone oils are high molecular weight synthetic polymeric compounds in which silicon atoms are joined in a chain-like and/or reticular manner via oxygen atoms and the remaining valences of the silicon are saturated by hydrocarbon radicals (in most cases methyl, less often ethyl, propyl, phenyl groups etc.). Systematically, the silicone oils are referred to as polyorganosiloxanes. The methyl-substituted polyorganosiloxanes, which are the most important compounds of this group in terms of amount and are characterized by the following structural formula 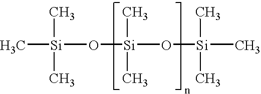
are also referred to as polydimethylsiloxane or Dimethicone (INCI). Dimethicones have various chain lengths and various molecular weights.

Particularly advantageous polyorganosiloxanes for the purposes of the present invention are, for example, dimethylpolysiloxanes [poly(dimethylsiloxane)], which are available, for example, under the trade names Abil 10 to 10 000 from Th. Goldschmidt. Also advantageous are phenylmethylpolysiloxanes (INCI: Phenyl Dimethicone, Phenyl Trimethicone), cyclic silicones (octamethylcyclotetrasiloxane and decamethylcyclopentasiloxane), which are also referred to in accordance with INCI as Cyclomethicone, amino-modified silicones (INCI: Amodimethicone) and silicone waxes, e.g. polysiloxane-polyalkylene copolymers (INCI: Stearyl Dimethicone and Cetyl Dimethicone) and dialkoxydimethylpolysiloxanes (Stearoxy Dimethicone and Behenoxy Stearyl Dimethicone), which are available as various Abil wax grades from Th. Goldschmidt. However, other silicone oils can also be used advantageously for the purposes of the present invention, for example cetyldimethicone, hexamethylcyclotrisiloxane, polydimethylsiloxane, poly(methylphenylsiloxane).

The preparations according to the present invention can also advantageously comprise one or more substances from the following group of siloxane elastomers, for example in order to increase the water resistance and/or the light protection factor of the products:
(a) siloxane elastomers which contain the units R₂SiO and RSiO_{1.5} and/or R₃SiO_{0.5} and/or SiO₂,
   where the individual radicals R, in each case independently of one another, are hydrogen, C₁₋₂₄-alkyl (such as, for example, methyl, ethyl, propyl) or aryl (such as, for example, phenyl or tolyl), alkenyl (such as, for example, vinyl), and the weight ratio of the units R₂SiO to RSiO_{1.5} is chosen from the range from 1:1 to 30:1;
(b) siloxane elastomers which are insoluble and swellable in silicone oil and which are obtainable by the addition reaction of an organopolysiloxane (1) which contains silicon-bonded hydrogen with an organopolysiloxane (2) which contains unsaturated aliphatic groups,
   where the quantitative amounts used are chosen such that the amount of hydrogen in the organopolysiloxane (1) or in the unsaturated aliphatic groups of the organopolysiloxane (2)
      is in the range from 1 to 20 mol % when the organopolysiloxane is non-cyclic and
      is in the range from 1 to 50 mol % when the organopolysiloxane is cyclic.

For the purposes of the present invention, the siloxane elastomer or elastomers are advantageously present in the form of spherical powders or in the form of gels.

Siloxane elastomers present in the form of spherical powders which are advantageous according to the invention are those with the INCI name Dimethicone/Vinyl Dimethicone Crosspolymer, for example that available from DOW CORNING under the trade names DOW CORNING 9506 Powder.

It is particularly preferred when the siloxane elastomer is used in combination with oils from hydrocarbons of animal and/or vegetable origin, synthetic oils, synthetic esters, synthetic ethers or mixtures thereof.

It is very particularly preferred when the siloxane elastomer is used in combination with unbranched silicone oils which are liquid or pasty at room temperature or cyclic silicone oils or mixtures thereof. Organopolysiloxane elastomers with the INCI name Dimethicone/Polysilicone-11, very particularly the Gransil grades obtainable from Grant Industries Inc. GCM, GCM-5, DMG-6, CSE gel, PM-gel, LTX, ININ gel, AM-18 gel and/or DMCM-5, are particularly advantageous.

It is very extremely preferred when the siloxane elastomer is used in the form of a gel of siloxane elastomer and a lipid phase where the content of the siloxane elastomer in the gel is 1 to 80% by weight, preferably 0.1 to 60% by weight, in each case based on the total weight of the gel.

It is advantageous for the purposes of the present invention to choose the total amount of the siloxane elastomers (active content) from the range from 0.01 to 10% by weight, advantageously from 0.1 to 5% by weight, in each case based on the total weight of the formulation.

The cosmetic and dermatological preparations according to the invention can comprise dyes and/or color pigments, particularly when they are in the form of decorative cosmetics. The dyes and color pigments can be chosen from the corresponding positive list in the Cosmetics Directive or the EC list of cosmetic colorants. In most cases, they are identical to dyes approved for foods. Advantageous colour pigments are, for example, titanium dioxide, mica, iron oxides (e.g. Fe₂O₃, Fe₃O₄, FeO(OH)) and/or tin oxide. Advantageous dyes are, for example, carmine, Prussian blue, chromium oxide green, ultramarine blue and/or manganese violet. It is particularly advantageous to choose the dyes and/or the color pigments from the Rowe Colour Index, 3rd Edition, Society of Dyers and Colourists, Bradford, England, 1971.

If the formulations according to the invention are in the form of products which are used on the face, it is favorable to choose one or more substances from the following group as the dye: 2,4-dihydroxyazobenzene, 1-(2′-chloro-4′-nitro-1′-phenylazo)-2-hydroxynaphthalene, Ceres red, 2-(sulfo-1-naphthylazo)-1-naphthol-4-sulfonic acid, calcium salt of 2-hydroxy-1,2′-azonaphthalene-1′-sulfonic acid, calcium and barium salts of 1-(2-sulfo-4-methyl-1-phenylazo)-2-naphthylcarboxylic acid, calcium salt of 1-(2-sulfo-1-naphthylazo)-2-hydroxynaphthalene-3-carboxylic acid, aluminum salt of 1-(4-sulfo-1-phenylazo)-2-naphthol-6-sulfonic acid, aluminum salt of 1-(4-sulfo-1-naphthylazo)-2-naphthol-3,6-disulfonic acid, 1-(4-sulfo-1-naphthylazo)-2-naphthol-6,8-disulfonic acid, aluminum salt of 4-(4-sulfo-1-phenylazo)-1-(4-sulfophenyl)-5-hydroxypyrazolone-3-carboxylic acid, aluminum and zirconium salts of 4,5-dibromofluorescein, aluminum and zirconium salts of 2,4,5,7-tetrabromofluorescein, 3′,4′,5′,6′-tetrachloro-2,4,5,7-tetrabromofluorescein and its aluminum salt, aluminum salt of 2,4,5,7-tetraiodofluorescein, aluminum salt of quinophthalonedisulfonic acid, aluminum salt of indigodisulfonic acid, red and black iron oxide (CIN: 77 491 (red) and 77 499 (black)), iron oxide hydrate (CIN: 77 492), manganese ammonium diphosphate and titanium dioxide.

Also advantageous are oil-soluble natural dyes, such as, for example, paprika extracts, β-carotene or cochineal.

Also advantageous for the purposes of the present invention are formulations with a content of pearlescent pigments. Preference is given in particular to the types of pearlescent pigments listed below:
1. Natural pearlescent pigments, such as, for example,
   “pearl essence” (guanine/hypoxanthin mixed crystals from fish scales) and
   “mother-of-pearl” (ground mussel shells)
2. Monocrystalline pearlescent pigments, such as, for example, bismuth oxychloride (BiOCl)
3. Layer-substrate pigments: e.g. mica/metal oxide

Bases for pearlescent pigments are, for example, pulverulent pigments or castor oil dispersions of bismuth oxychloride and/or titanium dioxide, and bismuth oxychloride and/or titanium dioxide on mica. The luster pigment listed under CIN 77163, for example, is particularly advantageous.

Also advantageous are, for example, the following types of pearlescent pigments based on mica/metal oxide:

| Group | Coating/layer thickness | Colour |
|---|---|---|
| Silver-white pearlescent | TiO₂: 40-60 nm | Silver |
| pigments | | |
| Interference pigments | TiO₂: 60-80 nm | Yellow |
| | TiO₂: 80-100 nm | Red |
| | TiO₂: 100-140 nm | Blue |
| | TiO₂: 120-160 nm | Green |
| Colour luster pigments | Fe₂O₃ | Bronze |
| | Fe₂O₃ | Copper |
| | Fe₂O₃ | Red |
| | Fe₂O₃ | Red-violet |
| | Fe₂O₃ | Red-green |
| | Fe₂O₃ | Black |
| Combination pigments | TiO₂/Fe₂O₃ | Gold shades |
| | TiO₂/Cr₂O₃ | Green |
| | TiO₂/Prussian blue | Deep blue |
| | TiO₂/carmine | Red |

Particular preference is given, for example, to the pearlescent pigments obtainable from Merck under the trade names Timiron, Colorona or Dichrona.

The list of given pearlescent pigments is not of course intended to be limiting. Pearlescent pigments which are advantageous for the purposes of the present invention are obtainable by numerous methods known per se. For example, other substrates apart from mica can be coated with further metal oxides, such as, for example, silica and the like. SiO₂ particles coated with, for example, TiO₂ and Fe₂O₃ (“ronaspheres”), which are sold by Merck and are particularly suitable for the optical reduction of fine lines, are advantageous.

It can, moreover, be advantageous to dispense completely with a substrate such as mica. Particular preference is given to iron pearlescent pigments prepared without the use of mica. Such pigments are obtainable, for example, under the trade name Sicopearl Kupfer 1000 from BASF.

In addition, also particularly advantageous are effect pigments which are obtainable under the trade name Metasomes Standard/Glitter in various colours (yellow, red, green, blue) from Flora Tech. The glitter particles are present here in mixtures with various auxiliaries and dyes (such as, for example, the dyes with the Colour Index (CI) numbers 19140, 77007, 77289, 77491).

The dyes and pigments may be present either individually or in a mixture, and can be mutually coated with one another, different coating thicknesses generally giving rise to different colour effects. The total amount of dyes and colour-imparting pigments is advantageously chosen from the range from, for example, 0.1% by weight to 30% by weight, preferably from 0.5 to 15% by weight, in particular from 1.0 to 10% by weight, in each case based on the total weight of the preparations.

The Pickering emulsions according to the invention can be used as bases for cosmetic or dermatological formulations. These can have the customary composition and be used, for example, for the treatment and care of the skin, as lipcare product, as deodorant product and as make-up or make-up removal product in decorative cosmetics or as sunscreen preparation. For use, the cosmetic and dermatological preparations according to the invention are applied to the skin in an adequate amount in the manner customary for cosmetics.

Accordingly, cosmetic or topical dermatological compositions for the purposes of the present invention can, depending on their formulation, be used, for example, as skin protection cream, cleansing milk, sunscreen lotion, nourishing cream, day or night cream etc. It is in some cases possible and advantageous to use the compositions according to the invention as bases for pharmaceutical formulations.

The cosmetic and dermatological preparations according to the invention can comprise cosmetic auxiliaries as are customarily used in such preparations, e.g. preservatives, preserving aids, complexing agents, bactericides, perfumes, substances for preventing or increasing foaming, dyes, pigments which have a coloring effect, thickeners, moisturizing and/or humectant substances, fillers which improve the skin feel, fats, oils, waxes or other customary constituents of a cosmetic or dermatological formulation, such as alcohols, polyols, polymers, foam stabilizers, electrolytes, organic solvents or silicone derivatives.

Advantageous preservatives for the purposes of the present invention are, for example, formaldehyde donors (such as, for example, DMDM hydantoin, which is available, for example, under the trade name Glydant® from Lonza), iodopropyl butyl carbamates (e.g. those available under the trade names Glycacil-L, Glycacil-S from Lonza and/or Dekaben LMB from Jan Dekker), parabens (i.e. p-hydroxybenzoic alkyl esters, such as methyl-, ethyl-, propyl- and/or butylparaben), phenoxyethanol, ethanol, benzoic acid and the like. Usually, the preservative system according to the invention further advantageously also comprises preserving aids, such as, for example, octoxyglycerol, glycine soya etc.

Advantageous complexing agents for the purposes of the present invention are, for example, EDTA, [S,S]-ethylenediamine disuccinate (EDDS), which is available, for example, under the trade name Octaquest from Octel, pentasodium ethylenediamine-tetramethylenephosphonate, which is available, for example, under the trade name Dequest 2046 from Monsanto and/or iminodisuccinic acid which is available, inter alia, from Bayer AG under the trade names Iminodisuccinat VP OC 370 (about 30% strength solution) and Baypure CX 100 solid.

In particular, the Pickering emulsions according to the invention can also comprise antioxidants. According to the invention, favorable antioxidants which may be used are all antioxidants which are customary or suitable for cosmetic and/or dermatological applications.

The antioxidants are advantageously chosen from the group consisting of amino acids (e.g. glycine, histidine, tyrosine, tryptophan) and derivatives thereof, imidazoles (e.g. urocanic acid) and derivatives thereof, peptides, such as D,L-carnosine, D-carnosine, L-carnosine and derivatives thereof (e.g. anserine), carotenoids, carotenes (e.g. α-carotene, β-carotene, lycopene) and derivatives thereof, chlorogenic acid and derivatives thereof, lipoic acid and derivatives thereof (e.g. dihydrolipoic acid), aurothioglucose, propylthiouracil and other thiols (e.g. thioredoxin, glutathione, cysteine, cystine, cystamine and the glycosyl, N-acetyl, methyl, ethyl, propyl, amyl, butyl and lauryl, palmitoyl, oleyl, γ-linoleyl, choleseteryl and glyceryl esters thereof) and salts thereof, dilauryl thiodipropionate, distearyl thiodipropionate, thiodipropionic acid and derivatives thereof (esters, ethers, peptides, lipids, nucleotides, nucleosides and salts) and sulfoximine compounds (e.g. buthionine sulfoximines, homocysteine sulfoximine, buthionine sulfones, penta-, hexa-, heptathionine sulfoximine) in very low tolerated doses (e.g. pmol to μmol/kg), and also (metal) chelating agents (e.g. α-hydroxy fatty acids, palmitic acid, phytic acid, lactoferrin), α-hydroxy acids (e.g. citric acid, lactic acid, malic acid), humic acid, bile acid, bile extracts, bilirubin, biliverdin, EDTA, EGTA and derivatives thereof, unsaturated fatty acids and derivatives thereof (e.g. γ-linolenic acid, linoleic acid, oleic acid), folic acid and derivatives thereof, ubiquinone and ubiquinol and derivatives thereof, vitamin C and derivatives (e.g. ascorbyl palmitate, Mg ascorbyl phosphate, ascorbyl acetate), tocopherols and derivatives (e.g. vitamin E acetate), vitamin A and derivatives (vitamin A palmitate), and coniferyl benzoate of benzoil resin, rutinic acid and derivatives thereof, α-glycosylrutin, ferrulic acid, furfurylideneglucitol, carnosine, butylhydroxytoluene, butylhydroxyinzole, nordihydroguaiacic acid, nordihydroguaiaretic acid, trihydroxybutyrophenone, uric acid and derivatives thereof, mannose and derivatives thereof, zinc and derivatives thereof (e.g. ZnO, ZnSO₄), selenium and derivatives thereof (e.g. selenomethionine), stilbenes and derivatives thereof (e.g. stilbene oxide, trans-stilbene oxide) and the derivatives (salts, esters, ethers, sugars, nucleotides, nucleosides, peptides and lipids) of these specified active ingredients which are suitable according to the invention.

The amount of the abovementioned antioxidants (one or more compounds) in the preparations according to the invention is preferably 0.001 to 30% by weight, particularly preferably 0.05 to 20% by weight, in particular 0.1 to 10% by weight, based on the total weight of the preparation.

If vitamin E and/or derivatives thereof are the antioxidant or the antioxidants, it is advantageous to choose their respective concentrations from the range from 0.001 to 10% by weight, based on the total weight of the formulation.

If vitamin A or vitamin A derivatives, or carotenes or derivatives thereof are the antioxidant or the antioxidants, it is advantageous to choose their respective concentrations from the range from 0.001 to 10% by weight, based on the total weight of the formulation.

It is particularly advantageous if the cosmetic preparations according to the present invention comprise cosmetic or dermatological active ingredients, preferred active ingredients being antioxidants which can protect the skin against oxidative stress.

Further advantageous active ingredients for the purposes of the present invention are natural active ingredients and/or derivatives thereof, such as, for example, α-lipoic acid, phytoene, D-biotin, coenzyme Q10, α-glucosylrutin, carnitine, carnosine, natural and/or synthetic isoflavonoids, creatin, taurine and/or β-alanine, and 8-hexadecene-1,16-dicarboxylic acid (dioic acid, CAS Number 20701-68-2; provisional INCI name Octadecenedioic acid).

Formulations according to the invention which comprise, for example, known antiwrinkle active ingredients, such as flavone glycosides (in particular α-glucosylrutin), coenzyme Q10, vitamin A and/or derivatives and the like, are particularly advantageously suitable for the prophylaxis and treatment of cosmetic or dermatological changes in the skin, as arise, for example, during skin aging (such as, for example, dryness, roughness and formation of dryness, wrinkles, itching, reduced regreasing (e.g. after washing), visible vascular dilations (telangiectases, couperosis), slackness and formation of lines and wrinkles, local hyperpigmentation, hypopigmentation and incorrect pigmentation (e.g. age spots), increased susceptibility to mechanical stress (e.g. cracking) and the like). They are also advantageously suitable for countering the appearance of dry and/or rough skin.

The water phase of the preparations according to the present invention can advantageously comprise customary cosmetic auxiliaries, such as, for example, alcohols, in particular those of low carbon number, preferably ethanol and/or isopropanol, diols or polyols of low carbon number, and ethers thereof, preferably propylene glycol, glycerol, butylene glycol, ethylene glycol, ethylene glycol monoethyl or monobutyl ether, propylene glycol monomethyl, monoethyl or monobutyl ether, diethylene glycol monomethyl or monoethyl ether and analogous products, foam stabilizers polymers, and electrolytes.

In addition, the preparations according to the invention can advantageously also comprise self-tanning substances, such as, for example, dihydroxyacetone and/or melanin derivatives in concentrations of from 1% by weight to 8% by weight, based on the total weight of the preparation.

In addition, the preparations according to the present invention can advantageously also comprise repellants for protection against flies, ticks and spiders and the like. For example, N,N-diethyl-3-methylbenzamide (trade name: Metadelphene, “DEET”), dimethyl phthalate (trade name; Palatinol M, DMP), and in particular ethyl 3-(N-n-butyl-N-acetylamino)propionate (available under the trade name Insekt Repellent® 3535 from Merck) are advantageous. The repellents can either be used individually or in combination.

Moisturizer is the term used to refer to substances or mixtures of substances which impart to cosmetic or dermatological preparations the property, following application or distribution on the surface of the skin, of reducing moisture release by the horny layer (also called transepidermal water loss (TEWL)) and/or of positively influencing hydration of the horny layer. Advantageous moisturizers for the purposes of the present invention are, for example, glycerol, lactic acid and/or lactates, in particular sodium lactate, butylene glycol, propylene glycol, biosaccharide gum-1, glycine soya, ethylhexyloxyglycerol, pyrrolidonecarboxylic acid and uric acid. In addition, it is particularly advantageous to use polymeric moisturizers from the group of water-soluble and/or water-swellable and/or water-gellable polysaccharides. Hyaluronic acid, chitosan starch hydroxypropyltrimonium chloride and/or a fucose-rich polysaccharide, which is filed in the Chemical Abstracts under the registry number 178463-23-5 and which is available, for example, under the name Fucogel® 1000 from SOLABIA S.A., for example, are particularly advantageous. Moisturizers can advantageously also be used as antiwrinkle active ingredients for the prophylaxis and treatment of cosmetic or dermatological changes in the skin, as arise, for example, during skin ageing.

The cosmetic or dermatological preparations according to the invention can also advantageously, but not necessarily, comprise fillers, which, for example, further improve the sensory and cosmetic properties of the formulations and, for example, bring about or enhance a velvety or silky feel on the skin. Advantageous fillers for the purposes of the present invention are starch and starch derivatives (such as, for example, tapioca starch, distarch phosphate, aluminum and sodium starch octenylsuccinate and the like), pigments which have neither a primarily UV filter effect nor a coloring effect (such as, for example, boron nitride etc.) and/or Aerosils® (CAS No. 7631-86-9).

Also advantageous for the purposes of the present invention are preparations which are in the form of cleansing emulsions which although advantageously comprise no washing-active surfactants, can be used, for example, to remove makeup or as mild washing foam—if appropriate also for blemished skin. Such cleansing emulsions may advantageously also be used as so-called rinse off preparations which are rinsed off from the skin following application.

Cosmetic and dermatological preparations which are in the form of a sunscreen composition are also favorable. For the purposes of the present invention, it is also advantageous to provide cosmetic and dermatological preparations whose main purpose is not protection against sunlight, but which nevertheless have a content of further UV protection substances. Thus, for example, UV-A and/or UV-B filter substances are usually incorporated into daycreams or make-up products. UV protection substances, like antioxidants and, if desired, preservatives, also constitute effective protection of the preparations themselves against spoilage.

Accordingly, for the purposes of the present invention, the preparations preferably comprise at least one further UV-A, UV-B and/or broadband filter substance. The formulations can, but do not necessarily, optionally also comprise one or more organic and/or inorganic pigments as UV filter substances, which may be present in the water phase and/or the oil phase.

In addition, the preparations according to the present invention can also advantageously be in the form of so-called oil-free cosmetic or dermatological emulsions, which comprise a water phase and at least one UV filter substance which is liquid at room temperature as a further phase, and which may particularly advantageously also be free from further oil components.

For the purposes of the present invention, particularly advantageous UV filter substances which are liquid at room temperature are homomenthyl salicylate (INCI: Homosalate), 2-ethylhexyl 2-cyano-3,3-diphenylacrylate (INCI: Octocrylene), 2-ethylhexyl 2-hydroxybenzoate (2-ethylhexyl salicylate, octyl salicylate, INCI: Ethylhexyl Salicylate) and esters of cinnamic acid, preferably 2-ethylhexyl 4-methoxycinnamate (INCI: Ethylhexyl Methoxycinnamate) and isopentyl 4-methoxycinnamate (INCI: Isoamyl p-Methoxycinnamate), 3-(4-(2,2-bis-ethoxycarbonylvinyl)phenoxy)propenyl)methoxy-siloxane/dimethylsiloxane copolymer, which is available, for example, under the trade name Parsol® SLX from Hoffmann La Roche.

Preferred inorganic pigments are metal oxides and/or other metal compounds which are insoluble or sparingly soluble in water, in particular oxides of titanium (TiO₂), zinc (ZnO), iron (e.g. Fe₂O₃), zirconium (ZrO₂), silicon (SiO₂), manganese (e.g. MnO), aluminum (Al₂O₃), cerium (e.g. Ce₂O₃), mixed oxides of the corresponding metals, and mixtures of such oxides, and also the sulfate of barium (BaSO₄).

For the purposes of the present invention, the pigments may advantageously also be used in the form of commercially available oily or aqueous predispersions. Dispersion auxiliaries and/or solubility promoters may advantageously be added to these predispersions.

Further advantageous pigments are latex particles. Latex particles advantageous according to the invention are those described in the following specifications: U.S. Pat. No. 5,663,213 and EP 0 761 201. Particularly advantageous latex particles are those which are formed from water and styrene/acrylate copolymers and are available, for example, under the trade name “Alliance SunSphere” from Rohm & Haas.

Advantageous UV-A filter substances for the purposes of the present invention are dibenzoylmethane derivatives, in particular 4-(tert-butyl)-4′-methoxydibenzoyl-methane (CAS No. 70356-09-1), which is sold by Givaudan under the name Parsol® 1789 and by Merck under the trade name Eusolex® 9020.

Advantageous further UV filter substances for the purposes of the present invention are sulfonated, water-soluble UV filters, such as, for example:
phenylene-1,4-bis(2-benzimidazyl)-3,3′-5,5′-tetrasulfonic acid and its salts, particularly the corresponding sodium, potassium or triethanolammonium salts, in particular the phenylene-1,4-bis(2-benzimidazyl)-3,3′-5,5′-tetrasulfonic acid bis-sodium salt with the INCI name Disodium Phenyl Dibenzimidazole Tetrasulfonate (CAS No.: 180898-37-7), which is available, for example, under the trade name Neo Heliopan AP from Haarmann & Reimer;
salts of 2-phenylbenzimidazole-5-sulfonic acid, such as its sodium, potassium or its triethanolammonium salt, and the sulfonic acid itself with the INCI name Phenylbenzimidazole Sulfonic Acid (CAS No. 27503-81-7), which is available, for example, under the trade name Eusolex 232 from Merck, or under Neo Heliopan Hydro from Haarmann & Reimer;
1,4-di(2-oxo-10-sulfo-3-bornylidenemethyl)benzene (also: 3,3′-(1,4-phenylene-dimethylene)bis(7,7-dimethyl-2-oxobicyclo[2.2.1]hept-1-ylmethanesulfonic acid) and salts thereof (particularly the corresponding 10-sulfato compounds, in particular the corresponding sodium, potassium or triethanolammonium salt), which is also referred to as benzene-1,4-di(2-oxo-3-bornylidenemethyl-10-sulfonic acid). Benzene-1,4-di(2-oxo-3-bornylidenemethyl-10-sulfonic acid) has the INCI name Terephthalidene Dicamphor Sulfonic Acid (CAS No.: 90457-82-2) and is available, for example, under the trade name Mexoryl SX from Chimex;
sulfonic acid derivatives of 3-benzylidenecamphor, such as, for example, 4-(2-oxo-3-bornylidenemethyl)benzenesulfonic acid, 2-methyl-5-(2-oxo-3-bornylidene-methyl)sulfonic acid and salts thereof.

Further advantageous UV filter substances for the purposes of the present invention are benzoxazole derivatives which are characterized by the following structural formula, 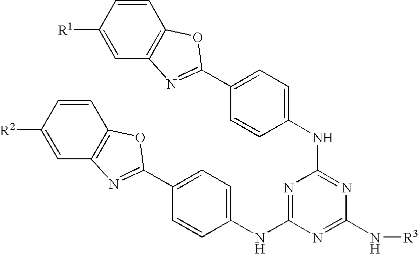
in which R¹, R² and R³, independently of one another, are chosen from the group of branched or unbranched, saturated or unsaturated alkyl radicals having 1 to 10 carbon atoms. It is particularly advantageous according to the invention to choose the radicals R¹ and R² to be the same, in particular from the group of branched alkyl radicals having 3 to 5 carbon atoms. It is also particularly advantageous for the purposes of the present invention if R³ is an unbranched or branched alkyl radical having 8 carbon atoms, in particular the 2-ethylhexyl radical.

A benzoxazole derivative which is particularly preferred according to the invention is 2,4-bis[5-1(dimethylpropyl)benzoxazol-2-yl(4-phenyl)imino]-6-(2-ethylhexyl)imino-1,3,5-triazine with the CAS No. 288254-16-0, which is characterized by the structural formula 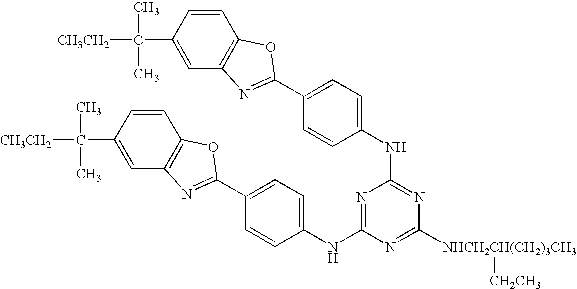
and is available from 3V Sigma under the trade name Uvasorb® K2A.

The benzoxazole derivative or derivatives are advantageously present in the cosmetic preparations according to the invention in dissolved form. In some circumstances, however, it may also be advantageous if the benzoxazole derivative or derivatives are present in pigmentary, i.e. undissolved, form—for example in particle sizes of from 10 nm to 300 nm.

Further advantageous UV filter substances for the purposes of the present invention are so-called hydroxybenzophenones. Hydroxybenzophenones are characterized by the following structural formula: 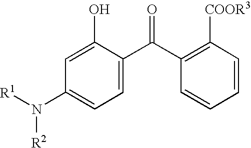
in which
R¹ and R², independently of one another, are hydrogen, C₁-C₂₀-alkyl, C₃-C₁₀-cycloalkyl or C₃-C₁₀-cycloalkenyl, where the substituents R¹ and R², together with the nitrogen atom to which they are bonded, can form a 5-membered or 6-membered ring and
R³ is a C₁-C₂₀-alkyl radical.

A particularly advantageous hydroxybenzophenone for the purposes of the present invention is hexyl 2-(4′-diethylamino-2′-hydroxybenzoyl)benzoate (also: Aminobenzophenone), which is characterized by the following structure: 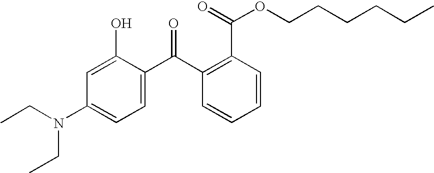
and is available under Uvinul A Plus from BASF.

Advantageous UV filter substances for the purposes of the present invention are also so-called broadband filters, i.e. filter substances which absorb both UV-A and also UV-B radiation.

Advantageous broadband filters or UV-B filter substances are, for example, triazine derivatives, such as, for example,
2,4-bis{[4-(2-ethylhexyloxy)-2-hydroxy]phenyl}-6-(4-methoxyphenyl)-1,3,5-triazine (INCI: Bis-Ethylhexyloxyphenol Methoxyphenyl Triazine), which is available under the trade name Tinosorb® S from CIBA-Chemikalien GmbH;
dioctylbutylamidotriazone (INCI: Diethylhexyl Butamido Triazone), which is available under the trade name UVASORB HEB from Sigma 3V;
Tris(2-ethylhexyl) 4,4′,4″-(1,3,5-triazine-2,4,6-triyltriimino)trisbenzoate, also: 2,4,6-tris[anilino(p-carbo-2′-ethyl-1′-hexyloxy)]-1,3,5-triazine (INCI: Ethylhexyl Triazone), which is sold by BASF Aktiengesellschaft under the trade name UVINUL® T 150;
2-[4,6-bis(2,4-dimethylphenyl)-1,3,5-triazin-2-yl]-5-(octyloxy)phenol (CAS No.: 2725-22-6).

An advantageous broadband filter for the purposes of the present invention is also 2,2′-methylenebis(6-(2H-benzotriazol-2-yl)-4-(1,1,3,3-tetramethylbutyl)phenol), (INCI: Methylene Bis-Benztriazolyl Tetramethylbutylphenol) which is available, for example, under the trade name Tinosorb® M from CIBA-Chemikalien GmbH.

For the purposes of the present invention, an advantageous broadband filter is also 2-(2H-benzotriazol-2-yl)-4-methyl-6-[2-methyl-3-[1,3,3,3-tetramethyl-1-[(trimethylsilyl)oxy]disiloxanyl]propyl]phenol (CAS No.: 155633-54-8) with the INCI name Drometrizole Trisiloxane.

The further UV filter substances may be oil-soluble or water-soluble. Advantageous oil-soluble filter substances are, for example:
3-benzylidenecamphor derivatives, preferably 3-(4-methylbenzylidene)camphor, 3-benzylidenecamphor;
4-aminobenzoic acid derivatives, preferably 2-ethylhexyl 4-(dimethyl-amino)benzoate, amyl 4-(dimethylamino)benzoate;
2,4,6-trianilino(p-carbo-2′-ethyl-1′-hexyloxy)-1,3,5-triazine;
esters of benzalmalonic acid, preferably di(2-ethylhexyl) 4-methoxybenzalmalonate;
esters of cinnamic acid, preferably 2-ethylhexyl 4-methoxycinnamate, isopentyl 4-methoxycinnamate;
derivatives of benzophenone, preferably 2-hydroxy-4-methoxybenzophenone, 2-hydroxy-4-methoxy-4′-methylbenzophenone, 2,2′-dihydroxy-4-methoxy-benzophenone and
UV filters bonded to polymers.

Advantageous water-soluble filter substances are, for example: Sulfonic acid derivatives of 3-benzylidenecamphor, such as, for example, 4-(2-oxo-3-bornylidenemethyl)benzenesulfonic acid, 2-methyl-5-(2-oxo-3-bomylidenemethyl)sulfonic acid and salts thereof.

A further light protection filter substance to be used advantageously according to the invention is ethylhexyl 2-cyano-3,3-diphenylacrylate (octocrylene), which is available from BASF under the name Uvinul® N 539 T.

Besides the filter substance(s) according to the invention, particularly advantageous preparations for the purposes of the present invention which are characterized by high or very high UV-A protection preferably also comprise further UV-A and/or broadband filters, in particular dibenzoylmethane derivatives [for example 4-(tert-butyl)-4′-methoxydibenzoylmethane] and/or 2,4-bis{[4-(2-ethylhexyloxy)-2-hydroxy]phenyl}-6-(4-methoxyphenyl)-1,3,5-triazine and/or phenylene-1,4-bis(2-benzimidazyl)-3,3′-5,5′-tetrasulfonic acid bis-sodium salt, in each case individually or in any combinations with one another. The list of given UV filters which can be used for the purposes of the present invention is not of course intended to be limiting.

The preparations according to the present invention advantageously comprise the substances which absorb UV radiation in the UV-A and/or UV-B region in a total amount of, for example, 0.1% by weight to 30% by weight, preferably 0.5 to 20% by weight, in particular 1.0 to 15.0% by weight, in each case based on the total weight of the preparations in order to provide cosmetic preparations which protect the hair and/or the skin from the entire range of ultraviolet radiation.

Preparations according to the invention can also advantageously be used as bases for cosmetic deodorants and/or antiperspirants, meaning that the present invention relates, in a particular embodiment, to Pickering emulsions as a basis for cosmetic deodorants.

In so-called antiperspirants, astringents—primarily aluminum salts, such as aluminum hydroxychloride (aluchlorhydrate)—reduces the formation of perspiration. In addition, the combination of astringents with antimicrobially effective substances in one and the same composition is customary. Antiperspirants are also advantageous for the purposes of the present invention.

The list of specified active ingredients and active ingredient combinations is not of course intended to be limiting.

The amount of antiperspirant active ingredients or deodorants (one or more compounds) in the preparations is preferably 0.01 to 30% by weight, particularly preferably 0.1-20% by weight, in particular 1-10% by weight, based on the total weight of the preparation.

According to the invention, the Pickering emulsions according to the invention are preferably used as impregnation solutions in combination with “dry” wipes. The wipes can consist of water-soluble, but advantageously of water-insoluble, materials. In addition, the wipes for the purposes of the present invention may be smooth, perforated or surface-structured. For the purposes of the present invention, wipes which consist of nonwoven material, in particular of water-jet-consolidated and/or water-jet-embossed nonwovens.

Such nonwovens can have macro embossing in any desired pattern. The choice which has to be made is governed firstly by the impregnation to be applied and secondly by the field of use in which the subsequent wipe is to be used.

If embossed nonwovens are used, then large cavities on the surface of the nonwoven and within the nonwoven facilitate the absorption of soil and impurities when the impregnated wipe is passed over the skin. The cleansing effect can be increased many times compared with unembossed wipes.

It has proven advantageous for the wipe if it has a weight of from 35 to 120 g/m², preferably from 40 to 60 g/m² (measured at 20° C.±2° C. and at an atmospheric humidity of 65% ±5% for 24 hours).

The thickness of the nonwoven is preferably 0.4 mm to 2 mm, in particular 0.6 mm to 0.9 mm.

Starting materials for the nonwoven of the wipe which may be used are generally all organic and inorganic natural- and synthetic-based fiber substances. By way of example, viscose, cotton, cellulose, jute, hemp, sisal, silk, wool, polypropylene, polyester, polyethylene terephthalate (PET), aramid, nylon, polyvinyl derivatives, polyurethanes, polylactide, polyhydroxyalkanoate, cellulose ester and/or polyethylene, and also mineral fibers such as glass fibers or carbon fibers may be listed. However, the present invention is not limited to the specified materials, and a large number of other fibers can be used to form the nonwoven. It is particularly advantageous for the purposes of the present invention if the fibers used are not water-soluble.

In a particularly advantageous embodiment of the nonwoven, the fibers consist of a mixture 70% by weight of viscose and 30% by weight of PET. In addition, preference is given to nonwovens of PET, viscose and cotton, for example those of 40% by weight PET, 50% by weight and 10% by weight cotton or those of 50% by weight PET, 40% by weight and 10% by weight cotton.

Fibers of high-strength polymers, such as polyamide, polyester and/or highly stretched polyethylene are particularly advantageous.

Moreover, the fibers can also be colored in order to be able to emphasize and/or increase the visual attractiveness of the nonwoven. The fibers can additionally comprise UV stabilizers and/or preservatives.

The fibers used to form the wipe preferably have a water-absorption rate of more than 60 mm/[10 min] (measured using the EDANA test 10.1-72), in particular more than 80 mm/[10 min].

In addition, the fibers used to form the wipe preferably have a water-absorption capacity of more than 5 g/g (measured using the EDANA test 10.1-72), in particular more than 8 g/g.

Advantageous wipes for the purposes of the present invention have a tear strength of in particular

| | | [N/50 mm] |
|---|---|---|
| In the dry state | Machine direction | >60, preferably >80 |
| | Transverse direction | >20, preferably >30 |
| In the impregnated state | Machine direction | >4, preferably >60 |
| | Transverse direction | >10, preferably >20 |

The expandability of advantageous wipes is preferably

| | | |
|---|---|---|
| In the dry state | Machine direction | 15% to 100%, preferably |
| | | 20% and 50% |
| | Transverse direction | 40% to 120%, preferably |
| | | 50% and 85% |
| In the impregnated state | Machine direction | 15% to 100%, preferably |
| | | 20% and 40% |
| | Transverse direction | 40% to 120%, preferably |
| | | 50% and 85% |

Advantageous impregnation solutions according to the invention have a viscosity of less than 2000 mPa·s. Such impregnation solutions can be applied to the wipe in a manner known per se.

According to the invention, not only are moist-impregnated wipes advantageous, but also so-called dry wipes, which are obtainable, for example, by finally drying moist-impregnated wipes. These wipes can be made ready for use again by the user by simple moistening.

The use of the wipes according to the invention for the cleansing and care of the skin and the use of wipes according to the invention as face wipes, baby wipes, intimate care wipes, deodorant wipes, sunscreen wipes and/or wipes for the care and/or cleansing of sensitive skin is also in accordance with the invention.

A particularly advantageous aspect of the low-viscosity emulsions according to the invention is that with their help pigments such as, for example, ZnO, can be applied to a wipe. The application of zinc oxide is particularly preferred if the wipes according to the invention are to be used in babycare. By applying ZnO, which is known for its antibacterial properties, additional protection against diaper dermatitis is achieved during daily cleaning.

The examples below are intended to illustrate the present invention without limiting it. The numerical values in the examples are percentages by weight, based on the total weight of the particular preparations.

### EXAMPLES

### Example 1

The primary emulsion 1 with a viscosity of >8000 mPa consisting of

| | Primary emulsion 1 |
|---|---|
| Titanium dioxide (Eusolex T2000) | 2.5 |
| Zinc oxide | 4 |
| Distarch phosphate | 1.5 |
| Hydrogenated coco glycerides | 1 |
| C₁₆₋₃₈ alkyl hydroxystearoyl stearate (Kester wax | 0.5 |
| K80P) | |
| Cetyldimethicone (Abil wax 9840) | 0.5 |
| Octyldodecanol | 3.75 |
| Butylene glycol caprylate/caprate | 5 |
| PVP/hexadecane copolymer | 0.5 |
| Stearyl alcohol | 1.5 |
| Cyclomethicone | 2 |
| Tocopheryl acetate | 0.5 |
| PPG-15 stearyl ether | 1 |
| Dicaprylyl ether (Cetiol OE) | 3.5 |
| Ethylhexyl methoxycinnamate | 5 |
| Bis-ethylhexyloxyphenol methoxyphenyltriazine | 2 |
| Octocrylene | 3.5 |
| Aminobenzophenone | 2.5 |
| Ethylhexyltriazone | 2 |
| Dronetrizole trisiloxane | 1.5 |
| Phenyldibenzimidazoltetrasulfonic acid | 2 |
| Preservative | 1 |
| Glycerol | 5 |
| NaOH 45% strength solution in water | 0.5 |
| EDTA solution | 1 |
| Perfume | 0.4 |
| Water | ad 70 |

is mixed with dilution solution 1 consisting of:

| | Dilution solution 1 |
|---|---|
| Hydroxyethylcellulose | 1 |
| Polyquaternium 37 | 0.1 |
| Water | 200 |

with stirring for 5 minutes using paddle stirrers at a stirring speed of 100 rpm.

### Example 2

The primary emulsion 2 with a viscosity of >8000 mPa consisting of:

| | Primary emulsion 2 |
|---|---|
| Titanium dioxide (Eusolex T2000) | 2 |
| Talc (talc micron) | 1 |
| Distarch phosphate | 1 |
| Tapioca starch | 3 |
| C₂₀₋₄₀ alkyl stearate (Kester wax K82) | 0.5 |
| Behenoxydimethicone (Abil wax 2440) | 1 |
| Polyisobutene (Rewopal PIB 1000) | 0.5 |
| Caprylic/capric triglyceride | 5 |
| Octyldodecanol | 5 |
| C₁₂₋₁₅ alkyl benzoate | 5 |
| Acetylated glycol stearate + tristearin | 2 |
| Stearyl alcohol | 1 |
| Dimethicone | 2 |
| Tocopheryl acetate | 1 |
| Starch hydroxypropyltrimonium chloride (Sensomer | 1 |
| Cl 50) | |
| Ethylhexyl methoxycinnamate | 5 |
| Bis-ethylhexyloxyphenol methoxyphenyltriazine | 1.5 |
| Methylenebisbenzotriazolyl tetramethylbutylphenol | 1 |
| Octocrylene | 3 |
| Terephthalidenedicamphorsulfonic acid | 0.5 |
| Dronetrizole trisiloxane | 1 |
| Dihydroxyacetone | 2.5 |
| Benzoxazole | 0.5 |
| Glycerol | 5 |
| Biosaccharide gel (Fucogel 1000) | 0.5 |
| Hyaluronic acid | 0.5 |
| EDTA solution | 1 |
| Water-soluble dye | 0.3 |
| Alcohol | 15 |
| Perfume | 0.3 |
| Water | ad 70 |

is mixed with dilution solution 2 consisting of:

| | Dilution solution 2 |
|---|---|
| Hydroxypropylmethylcellulose | 2 |
| Polyquaternium 37 | 1 |
| Water | 300 |

with vigorous shaking for 2 minutes.

### Example 3

The primary emulsion 3 with a viscosity of >8000 mPa consisting of:

| | Primary emulsion 3 |
|---|---|
| Titanium dioxide (Eusolex T2000) | 5 |
| Zinc oxide | 5 |
| Silica (Aerosil R972) | 5 |
| Preservative | 10 |
| Glycerol | 10 |
| Panthenol | 1 |
| Citric acid | 8 |
| Sodium citrate | 2 |
| Potassium sorbate | 1 |
| EDTA solution | 6 |
| Fat-soluble dye | 2 |
| Perfume | 5 |
| Water | ad 70 |

is mixed with dilution solution 3 consisting of:

| Dilution solution | 3 |
|---|---|
| Hydroxyethylcellulose | 6 |
| Polyvinylpyrrolidone | 2 |
| Water | ad 930 |

with stirring for 5 minutes using paddle stirrers at a stirring speed of 100 rpm.

Examples of low viscosity Pickering emulsions according to the invention which can be used directly are:

| | 4 | 5 | 6 | 7 | 8 |
|---|---|---|---|---|---|
| Titanium dioxide (Eusolex T2000) | 3 | 2 | 3 | 2 | 0.35 |
| Zinc oxide | | 4 | | | 0.65 |
| Titanium dioxide (titanium dioxide T805) | 3 | | | 3 | |
| Silica (Aerosil R972) | | | | 0.5 | |
| Talc (talc micron) | 2 | | | 2 | 0.5 |
| Boron nitride | | | | 1 | |
| Sodium corn starch n-octenyl succinate | | 1 | 0.5 | | |
| Distarch phosphate | | 1 | | | |
| Tapioca starch | 4 | 5 | | | |
| Hydrogenated coco glycerides | 1 | 1 | | | |
| C₁₆₋₃₈ alkyl hydroxystearoyl stearate (Kester | | | 3 | | |
| wax K80P) | | | | | |
| Behenoxydimethicone (Abil wax 2440) | | 1 | | | |
| Cetyldimethicone (Abil wax 9840) | | | | 1 | |
| Caprylic/capric triglyceride | | 4 | 8 | 5 | |
| Octyldodecanol | 4.25 | 4 | 8 | | |
| Mineral oil | 3.65 | 4 | 8 | | 8 |
| Butylene glycol caprylate/caprate | 5 | | | 5 | |
| C₁₂₋₁₅ alkyl benzoate | | | 5 | 3 | |
| PVP/hexadecene copolymer | 0.5 | | | 1 | |
| Acetylated glycol stearate + tristearin | | | | 0.5 | |
| Stearyl alcohol | 1.5 | 1 | | | |
| Dimethicone | | | | 2 | |
| Cyclomethicone | 2 | 3 | | | |
| Tocopheryl acetate | 0.5 | | | 1 | |
| Ubiquinone | | 0.01 | | | |
| PEG-40 hydrogenated castor oil | 1 | | | | |
| Dicaprylyl ether (Cetiol OE) | | | 5 | 3 | |
| Hydrogenated polyisobutene (Polysynlan) | | | | 0.1 | |
| Ethylhexyl methoxycinnamate | 5 | | | 4 | |
| Bisethylhexyloxyphenol | 1.7 | | | 1 | |
| methoxyphenyltriazine | | | | | |
| Methylenebisbenzotriazolyl | | | | 1 | |
| tetramethylbutylphenol | | | | | |
| Octocrylene | 3.5 | | | 2 | |
| Aminobenzophenone | | | | 0.5 | |
| Diethylhexyl naphthalate | | | 4 | | |
| Ethylhexyltriazone | | | 4 | | |
| Terephthalidenedicamphorsulfonic acid | | | 0.5 | | |
| Butylmethoxydibenzoylmethane | | | 2 | 2 | |
| Dihydroxyacetone | | 5 | | | |
| Diethylhexylbutamidotriazone (UVASORBHEB) | | | | 1 | |
| Benzoxazole | 1 | | | | |
| Phenyldibenzimidazoletetrasulfonic acid | | | 2 | 2 | |
| Phenylbenzimidazolesulfonic acid | | | | 2 | |
| Preservative | 1 | 1 | | | 0.6 |
| Glycerol | 7 | 3 | 5 | | 3 |
| Aluminum chlorohydrate | | | | | 10 |
| Panthenol | | 1 | | | |
| Hyaluronic acid | | | | 0.5 | |
| Chitosan | 0.5 | | | | 0.25 |
| Lactic acid | 0.3 | 0.8 | | | 0.3 |
| Citric acid | | | | | 0.09 |
| Sodium citrate | | | | | 0.18 |
| Potassium sorbate | | | | | 0.05 |
| NaCl | | | 0.9 | | |
| Carbomer (Carbopol 981) | | | 0.1 | | |
| C₁₀₋₃₀ alkyl acrylate crosspolymer (Pemulen | | | 0.3 | | |
| TR1) | | | | | |
| Dimethicone/polysilicone-11 | | | | 0.2 | |
| Xanthan gum | | | 0.1 | | |
| Hydroxyethylcellulose | 0.4 | | | | 0.4 |
| Hydroxypropylmethylcellulose | | 0.1 | | | 0.2 |
| Methylhydroxyethylcellulose | 0.1 | | | 0.8 | 0.1 |
| Polyquaternium 37 | | 2 | | | |
| Hydroxypropylstarch phosphate | | | 3 | | |
| Polyacrylate | | | | 0.1 | |
| NaOH 45% strength solution in water | | | 1.2 | 2 | |
| EDTA solution | 1 | | 1 | | |
| Water-soluble dye | | | | 0.5 | |
| Alcohol | | | 10 | 15 | |
| Perfume | 0.4 | | | | |
| Water | ad | ad | ad | ad | ad |
| | 100 | 100 | 100 | 100 | 100 |

1. A Pickering water-in-oil emulsion, said emulsion comprising
(a) an oil phase,
(b) a water phase,
(c) at least one type of microfine, inorganic metal oxide particles
1) having an average particle diameter of less than 200 nm,
2) exhibiting both hydrophilic and lipophilic properties, and
3) dispersible both in lipophilic and hydrophilic liquids,
(d) 0.01 to 5% by weight of at least one thickener selected from the group consisting of polymeric nonionic thickeners, polymeric cationic thickeners, and combinations thereof,
(e) up to 0.5% by weight of one or more emulsifiers, and
(f) a viscosity of less than 3000 mPa·s.

2. The Pickering emulsion as claimed in claim 1, wherein the emulsion is emulsifier-free.

3. The Pickering emulsion as claimed in claim 1, wherein the amount of the particles is between 1% by weight and 15% by weight of the emulsion.

4. The Pickering emulsion as claimed in claim 1, wherein the average particle size diameter of the particles is between 5 nm and 100 nm.

5. The Pickering emulsion as claimed in claim 1, wherein the at least one thickener includes hydroxyethylcellulose.

6. The Pickering emulsion as claimed in claim 1, wherein the at least one thickener includes polyquaternium 37.

7. The Pickering emulsion as claimed in claim 1, wherein the amount of the at least one thickener is between 0.05 and 3% by weight.

8. The Pickering emulsion as claimed in claim 1, further comprising at least one UV filter substance.

9. The Pickering emulsion as claimed in claim 1, wherein the particles are surface-coated.

10. A cosmetic or dermatological wipe, comprising a water-insoluble nonwoven which is moistened with a cosmetic or dermatological impregnation composition, the composition formed of a Pickering water-in-oil emulsion, comprising:
(a) an oil phase,
(b) a water phase,
(c) at least one type of microfine, inorganic metal oxide particles
1) having an average particle diameter of less than 200 nm,
2) exhibiting both hydrophilic and lipophilic properties, and
3) dispersible both in lipophilic and hydrophilic liquids,
(d) 0.01 to 5% by weight of at least one thickener selected from the group consisting of polymeric nonionic thickeners, polymeric cationic thickeners, and combinations thereof,
(e) up to 0.5% by weight of one or more emulsifiers, and
(f) a viscosity of less than 2000 mPa·s.

11. The wipe as claimed in claim 10, wherein the weight ratio of the unimpregnated wipe to the impregnation composition is from 1:1 to 1:5.

12. The wipe as claimed in claim 10, wherein the emulsion is emulsifier-free.

13. The wipe as claimed in claim 10, wherein the average particle size diameter of the particles is between 5 nm and 100 nm.

14. The wipe as claimed in claim 10, wherein the at least one thickener includes hydroxyethylcellulose.

15. The wipe as claimed in claim 10, wherein the at least one thickener includes polyquaternium 37.

16. The wipe as claimed in claim 10, further comprising at least one UV filter substance.

17. A method of preparing a Pickering emulsion with a viscosity of less than 3000 mPa·s, comprising the step of
mixing a primary emulsion having a viscosity of 8000 mPa·s or more and comprising
(a) an oil phase,
(b) a water phase,
(c) at least one type of microfine inorganic metal oxide particles
1) having an average particle size of less than 200 nm,
2) exhibiting both hydrophilic and lipophilic properties, and
3) dispersible both in hydrophilic and lipophilic media,
with an aqueous solution in which at least one thickener is present in an evenly distributed form.

18. The method of claim 17, wherein the emulsion is emulsifier-free.

19. The method of claim 17, wherein the average particle diameter of the particles is between 5 nm and 100 nm.

20. The method of claim 17, wherein the at least one thickener includes hydroxyethylcellulose.

21. The method of claim 17, wherein the at least one thickener includes polyquaternium 37.
